(19) United States
(12) Patent Application Publication
(10) Pub. No.: US 2025/0363661 A1
(43) Pub. Date: Nov. 27, 2025

(54) MACHINE LEARNING-BASED SHAPE ESTIMATION AND IMAGING FOR FLEXIBLE ULTRASOUND TRANSDUCER ARRAY

(71) Applicant: GaleSun Technologies, New York, NY (US)

(72) Inventors: Jason Mullins, Philadelpia, PA (US); Shristopher Khan, Franklin, TN (US)

(21) Appl. No.: 19/217,658

(22) Filed: May 23, 2025

Related U.S. Application Data
(60) Provisional application No. 63/651,054, filed on May 23, 2024.

(51) Int. Cl. G06T 7/73; G06T 7/00; G06T 7/11

(52) U.S. Cl. CPC G06T 7/73; G06T 7/0002; G06T 7/11; G06T 2207/10132; G06T 2207/20081; G06T 2207/20084; G06T 2207/30004; G06T 2207/30168; G06T 2207/30244

(57) ABSTRACT

A method for locating an unknown arrangement of ultrasound transducer elements includes acquiring a set of raw ultrasound data using a flexible or rigid array of transducer elements, extracting a set of features from the ultrasound data, the features being derived from fundamental physics of ultrasound propagation and structural characteristics of a target object being imaged, organizing and structuring the extracted features as input for a machine learning model, and determining spatial coordinates of each ultrasound transducer element based on an output of the machine learning model.

### CROSS-REFERENCE TO RELATED APPLICATION

This application claims the benefit of and priority to U.S. Provisional Patent Application No. 63/651,054 filed on May 23, 2024, the entire disclosure of which is hereby incorporated by reference in its entirety for all purposes.

### TECHNICAL FIELD

The present application relates to the field of ultrasound imaging systems, and more particularly to systems and methods for estimating the spatial configuration of flexible or stretchable ultrasound transducer elements using machine learning techniques.

### BACKGROUND

Ultrasound imaging is a widely utilized non-invasive technique for visualizing the internal structures of objects and biological tissues, where images are created by transmitting soundwaves into the object under investigation from one or multiple elements of a transducer array and subsequently recording the echoes reflected by the interior of the object. This recorded radiofrequency (RF) data is mapped to a two-dimensional (2D) space or three-dimensional (3D) space based on the time that it took for the echo to return and a known or assumed sound speed of the material in the object. Due to its noninvasiveness and real-time observation, ultrasound imaging is extensively employed in industrial and biomedical contexts to detect defects in solid materials, assess tissue integrity, and monitor physiological changes.

Traditional ultrasound systems use fixed arrays of transducer elements to emit and receive sound waves, with the spatial configuration of these elements being predefined and rigidly structured. However, in recent years, there has been an increasing interest in utilizing flexible or conformal ultrasound transducer arrays. These arrays may adapt to complex geometries and maintain contact with non-planar surfaces, making them ideal for dynamic imaging applications, such as monitoring of moving body parts or irregularly shaped industrial components, as well as for imaging geometrically complex objects where it is difficult to obtain and maintain sufficient contact between the transducer elements and the object being imaged. Conformal arrays may exhibit single-axis flexibility, multi-axis flexibility, or stretchability, allowing for spatial reconfiguration of the transducer elements during operation. These arrays are sometimes configured as single “patches” in a single location or multiple separate sets of elements located on multiple parts of the object to be examined.

Despite their potential, creating accurate ultrasound images from these flexible arrays presents significant challenges. Ultrasound imaging relies heavily on the accurate spatial positioning of transducer elements. Even minor discrepancies in the assumed location of the transducer elements may result in significant imaging artifacts due to destructive interference caused by misaligned data acquisition paths. A number of approaches have previously been proposed to perform element localization for flexible ultrasound arrays. However, each one has its own drawbacks as outlined below.

One approach for performing element localization in flexible ultrasound arrays involves the use of external hardware, such as optical tracking systems, optical fibers, or strain sensors. These devices provide positional information that may be used to determine the spatial configuration of transducer elements. However, the reliance on external hardware introduces significant drawbacks. The addition of such components increases the overall cost and complexity of the ultrasound system and may also interfere with certain imaging procedures. For instance, optical tracking systems require that the optical markers remain within the field of view of the tracking camera, limiting the feasibility of imaging during dynamic motion or when the transducer array must move relative to the object being examined. Despite the utility of these external systems, prior studies have shown that they often fail to achieve sufficient localization accuracy for use in human biological tissue, particularly in applications requiring flexible arrays.

Another method for element localization involves iteratively estimating the shape of the flexible array through optimization of an objective function based on ultrasound image quality metrics. Various metrics, such as image sharpness, brightness, entropy, and coherence, are employed to assess image quality and adjust the estimated positions of the transducer elements accordingly. While this method avoids the need for external hardware, it is computationally intensive due to the iterative nature of the optimization process. Moreover, the effectiveness of this approach depends heavily on the selection of an appropriate objective function. If the objective function lacks a well-defined convex or concave structure, the optimization may converge to local minima or maxima, resulting in suboptimal localization accuracy and unreliable image quality.

A further technique for determining element coordinates involves triangulation based on the time-of-flight (ToF) of acoustic wavefronts between transducer elements. In this method, a full matrix capture (FMC) sequence is performed, with each element sequentially transmitting a signal while all other elements receive it. The ToF data is then converted to distance measurements using an assumed sound speed for the imaging medium. An optimization algorithm adjusts the estimated coordinates to minimize the error between calculated and measured distances. This method has been shown to achieve high localization accuracy in homogeneous media, where the sound speed remains constant. However, in heterogeneous media such as human tissue, variations in sound speed can significantly degrade localization accuracy, limiting the method's effectiveness in practical applications.

Recently, machine learning techniques, specifically deep neural networks (DNNs), have been explored for estimating the shape of flexible ultrasound arrays. These networks are trained using synthetic and in vivo ultrasound data to predict either the final B-mode image or geometric parameters representing the transducer array's configuration. Despite the promise of this approach, relying solely on raw ultrasound RF data without targeted feature extraction can limit the model's generalizability across diverse imaging conditions. This is particularly problematic in heterogeneous tissues, where significant variations in acoustic properties can affect signal characteristics. Previous attempts to use DNNs for shape estimation have not yet achieved the level of accuracy and generalizability required for practical clinical or industrial use.

Therefore, there remains a need for a cost-effective, accurate, and generalizable method and system for determining the spatial configuration of ultrasound transducer elements in flexible or stretchable arrays without relying on external tracking hardware.

### SUMMARY

To address the aforementioned shortcomings, a method and system for locating an unknown arrangement of ultrasound transducer elements are provided. The method includes acquiring a set of raw ultrasound data using a flexible or rigid array of transducer elements, extracting a set of features from the ultrasound data, the features being derived from fundamental physics of ultrasound propagation and structural characteristics of a target object being imaged, organizing and structuring the extracted features as input for a machine learning model, and determining spatial coordinates of each ultrasound transducer element based on an output of the machine learning model.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features that will be more readily apparent from the detailed description, the appended claims, and the accompanying figures (or drawings). A brief introduction of the figures is below.

FIG. 1 is a block diagram of an example architecture of a machine learning-based ultrasound imaging system, according to some embodiments of the disclosure.

FIG. 2 illustrates an example arrangement and configuration of a flexible ultrasound transducer array, according to some embodiments of the disclosure.

FIG. 3 illustrates an example feature extraction process implemented by a feature engineering module, according to some embodiments of the disclosure.

FIG. 4 illustrates an example shape estimation process implemented by a DNN, according to some embodiments of the disclosure.

FIG. 5 illustrates example composite features incorporating previous shape estimate and image quality metrics from previous processed images, according to some embodiments of the disclosure.

FIG. 6 illustrates an example process of using spatial configuration estimates and acquired ultrasound data to reconstruct high-resolution images of a target object, according to some embodiments of the disclosure.

FIG. 7 is a flow chart of an example method for machine learning-based shape estimation of transducer elements, according to some embodiments of the disclosure.

FIGS. 8A-8C illustrate comparisons of various ultrasound images obtained based on the disclosed shape estimation and the known reference configuration, according to some embodiments of the disclosure.

FIG. 9 is a block diagram of an example computer for machine learning-based shape estimation and imaging, according to some embodiments of the disclosure.

### DETAILED DESCRIPTION

The figures (FIGS.) and the following description relate to some embodiments by way of illustration only. It is to be noted that from the following description, alternative embodiments of the structures and methods disclosed herein will be readily recognized as viable alternatives that may be employed without departing from the principles of the present disclosure.

Reference will now be made in detail to some specific embodiments, examples of which are illustrated in the accompanying figures. It is to be noted that wherever practicable similar or like reference numbers may be used in the figures and may indicate similar or like functionality. The figures depict embodiments of the disclosed system (or method) for illustration purposes only. One skilled in the art will readily recognize from the following description that alternative embodiments of the structures and methods illustrated herein may be employed without departing from the principles described herein.

### Motivation and Technical Benefits

Ultrasound imaging is a widely used diagnostic tool in both medical and industrial settings due to its non-invasive nature and capability to provide real-time visualization of internal structures. Traditional ultrasound systems typically employ fixed or rigid transducer arrays with predetermined spatial configurations. However, these systems face significant limitations when imaging surfaces with complex or dynamically changing geometries, such as body parts in motion or irregular industrial components. Flexible and stretchable transducer arrays have emerged as a promising solution to address these challenges, as they can conform to complex shapes and maintain optimal contact with the target surface. Despite their advantages, accurately determining the spatial configuration of such arrays without external tracking systems remains a significant challenge. Current methods either rely on external hardware, such as optical tracking systems or strain sensors, or require computationally intensive iterative optimization processes. These approaches not only increase the system's cost and complexity but also introduce potential sources of error due to calibration issues or hardware limitations. Machine learning techniques, specifically DNNs, have been explored for estimating the shape of flexible ultrasound arrays. However, these attempts to use DNNs for shape estimation have not yet achieved the level of accuracy and generalizability required for practical clinical or industrial use, as described earlier.

The present disclosure addresses these challenges and other problems described earlier in the existing flexible ultrasonic imaging by introducing a method and system for estimating the spatial configuration of flexible or stretchable ultrasound transducer arrays through machine learning-based processing of certain features derived from raw ultrasound data. The method includes transmitting ultrasound waves from one or more transducer elements and receiving the reflected echoes. These received signals are then converted to digital data and subjected to preprocessing steps, including frequency filtering and time gain compensation. Subsequently, spatial features such as time-of-flight, amplitude, cross-correlation and certain other features are extracted and used as input to a machine learning model, such as a DNN. The DNN is trained to infer the spatial coordinates of each transducer element, allowing the system to accurately determine the array configuration without relying on external tracking systems. This estimated configuration is then used in an ultrasound imaging pipeline to reconstruct images with high spatial fidelity. Additionally, the system dynamically adjusts the firing sequence and data acquisition parameters based on the estimated configuration, thus optimizing data collection in real time.

The disclosed method and system provide several technical advantages over existing methods for spatial configuration estimation of flexible ultrasound transducer arrays. A key benefit is the elimination of external tracking systems, which are typically required in the existing systems to monitor the spatial arrangement of the transducer elements. By extracting spatial features directly from the received ultrasound data and processing them through a machine learning model, the disclosed system reduces system cost, complexity, and potential calibration errors associated with external hardware components.

Moreover, the use of machine learning techniques improves the accuracy of shape estimation, particularly in complex imaging scenarios involving non-planar surfaces or dynamically moving objects. The proposed system processes features such as time-of-flight, amplitude, and cross-correlation, enabling precise determination of transducer positions based solely on the received ultrasound signals. This capability is particularly beneficial in medical applications where maintaining consistent contact between the rigid transducers and the target surface is challenging.

The system also incorporates adaptive imaging capabilities, allowing it to adjust data acquisition and processing parameters based on the estimated configuration of the transducer elements. This real-time adaptation optimizes image quality and ensures accurate spatial mapping even in scenarios involving rapid motion or changing surface geometries. Additionally, the implementation of targeted feature engineering techniques reduces computational overhead while enhancing the generalizability and robustness of the machine learning model.

Furthermore, the disclosed system is highly scalable and may be implemented across various transducer configurations, including linear, matrix, and stretchable arrays. This versatility expands its potential applications across diverse fields, ranging from medical diagnostics to industrial defect detection, thereby increasing its practical utility and commercial viability.

It is to be noted that the benefits and advantages described herein are not all-inclusive, and many additional features and advantages will be further described under the context of specific embodiments. In addition, some additional features and advantages will become apparent to one of ordinary skill in the art in view of the figures and the following descriptions.

### System Architecture

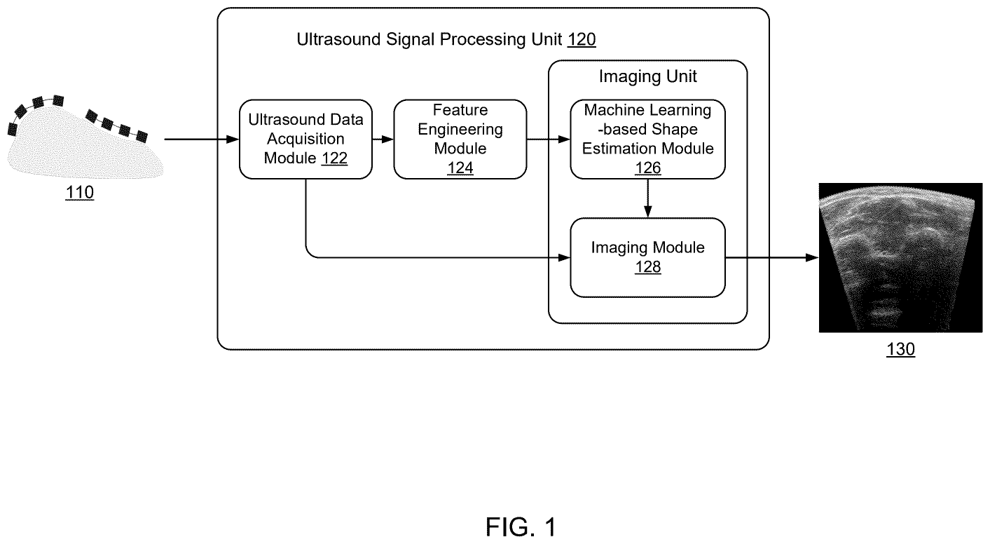

FIG. 1 is a block diagram of an example architecture of a machine learning-based ultrasound imaging system 100, according to some embodiments, according to some embodiments of the disclosure. As illustrated in the figure, the machine learning-based ultrasound imaging system 100 includes a flexible ultrasound transducer array 110, an ultrasound signal processing unit 120, and an output unit 130 for outputting or displaying the processed results. The ultrasound signal processing unit 120 optionally includes an ultrasound data acquisition module 122, a feature engineering module 124, a machine learning-based shape estimation module 126, and an imaging module 128. Each component plays a distinct yet interconnected role in achieving accurate spatial configuration estimation of the transducer elements in the array, and in obtaining high-resolution imaging in medical and industrial applications based on the accurate spatial configuration estimation of the transducer elements in the array. The specific functions of these components are further described in detail below.

The flexible ultrasound transducer array 110 is the foundational element of the system 100, comprising multiple transducer elements (or simply transducers) arranged in a flexible or stretchable matrix configuration. The transducers may be fabricated from piezoelectric materials that can both emit and receive ultrasound waves, allowing them to function in both transmit and receive modes. These elements may be embedded in a substrate that enables the array to conform to curved or irregular surfaces, such as human body parts or complex industrial components. The array may comprise a single patch of transducers or multiple patches that may be positioned independently to achieve comprehensive spatial coverage. The design allows for varying degrees of flexibility, ranging from single-axis bending to multi-axis deformation, where the transducer elements may be secured to the target surface using adhesives, straps, or other attachment mechanisms. This structural adaptability ensures optimal contact with dynamic surfaces, making the array particularly suited for applications involving movement or non-planar geometries.

The data acquisition module 122 is responsible for capturing and digitizing the ultrasound signals generated by the transducer array 110. According to some embodiments, the ultrasound transducer array 110 may include a signal transmitter that generates electrical signals, which are then converted into ultrasound waves by the transducer elements. Following transmission, the reflected echoes may be received and converted back into electrical signals by the receivers. These signals are subsequently digitized by an analog-to-digital converter (ADC) operating at a sampling rate selected to preserve the desired resolution and frequency range (typically between 1 MHz and 20 MHZ). The digital data may be acquired by the ultrasound data acquisition module 122, which may temporarily store the acquired data in a data buffer to ensure synchronized processing before being archived in a local storage unit or transmitted to an external computing system for further analysis. In some embodiments, the ultrasound data acquisition module 122 may be configured to handle multi-channel data collection, allowing for simultaneous acquisition from multiple transducer elements. This configuration enhances spatial resolution and provides comprehensive data for subsequent shape estimation and imaging processes. Once the ultrasound data has been acquired and digitized, it may be further processed in the feature engineering module 124 and the machine learning-based shape estimation module 126, as further described in detail below. In some embodiments, once the spatial configuration estimation of the transducer elements in the array is completed, the ultrasound data obtained from the transducer array 110 may be directly fed into the imaging module 128, which may perform an image reconstruction for the ultrasound data based on the determined spatial configuration estimation of the transducer elements in the array.

This feature engineering module 124 may begin the data processing by applying preprocessing steps to the raw data to reduce noise and enhance signal quality (the preprocessing steps may be also performed in the ultrasound data acquisition module 122, depending on the configuration of the system 100). Common preprocessing techniques include frequency filtering to eliminate unwanted components, time gain compensation to adjust signal amplitude based on propagation distance, and envelope extraction to isolate the amplitude envelope of the received signals. Following preprocessing, spatial features may be extracted to facilitate shape estimation. These features may include but are not limited to ToF data, which captures the time delay between transmitted and received signals to provide distance measurements, and amplitude data, which reflects signal intensity and surface reflectivity. Additionally, cross-correlation coefficients may be further calculated to assess the similarity between waveforms received by different transducer elements, aiding in the identification of relative positions of the transducer elements. The preprocessed data and extracted features may then be input into the machine learning-based shape estimation module 126.

The machine learning-based shape estimation module 126 may include a machine learning model configured for shape estimation. According to one embodiment, the machine learning model included in the shape estimation module 126 may be a DNN or the like. The DNN or other similar machine learning models may be trained using a combination of synthetic and real ultrasound data, covering a range of geometric configurations and tissue properties to enhance model robustness and generalizability. The network architecture of the DNN or other similar machine learning models may comprise multiple layers, including convolutional layers for spatial feature extraction and fully connected layers for coordinate mapping. Activation functions may be further employed to model complex, nonlinear relationships between features and spatial positions. The output of the DNN or other similar machine learning models is a set of coordinates representing the estimated positions or estimated shape of each transducer element in either 2D or 3D space. These estimated shapes of the transducer elements are subsequently passed to the imaging module 128 for image processing based on the estimated shape of the transducer elements.

The imaging module 128 may be configured to reconstruct an ultrasound image for a target object under examination based on the estimated spatial configuration of the transducer elements. The imaging module 128 (or the machine learning-based shape estimation module 126, depending on the configuration of the system 100) may refine the coordinates obtained from the machine learning model using optimization algorithms to minimize discrepancies between the predicted and actual distances. This step may ensure accurate spatial mapping, particularly in scenarios involving dynamic motion or complex geometries. The refined coordinates are then utilized in the image reconstruction process, which employs standard ultrasound imaging algorithms such as delay and sum (DAS) or delay multiply and sum (DMAS). These algorithms use the estimated spatial coordinates to generate 2D or 3D ultrasound images, which are further processed to enhance clarity and resolution through techniques such as log compression and envelope detection and the like.

In some embodiments, while not shown, the disclosed machine learning-based ultrasound imaging system 100 may optionally include an adaptive imaging control module that dynamically adjusts data acquisition parameters based on the estimated transducer configuration. This adaptive control ensures optimal data collection in scenarios involving rapid motion or changing surface geometries, such as imaging a moving organ or a deformable structure.

The output unit 130 may display the reconstructed image on a graphical user interface, allowing real-time visualization of the scanned object. In some embodiments, a same or another different user interface may also be utilized to display diagnostic information, including but are not limited to estimated transducer positions, shape metrics, and image quality indicators. In some embodiments, the estimated shape of the transducer elements may also be output through the output unit 130.

As can be seen above, through the integration of flexible transducer arrays, advanced data acquisition techniques, machine learning-driven feature extraction, and adaptive imaging control, accurate shape estimation and high-resolution imaging may be obtained through the disclosed system 100 without the need for external tracking hardware, which improves the performance of ultrasound imaging systems on object detection. The specific functions of the components in the system 100 are further described in detail below.

### Flexible Ultrasound Transducer Array

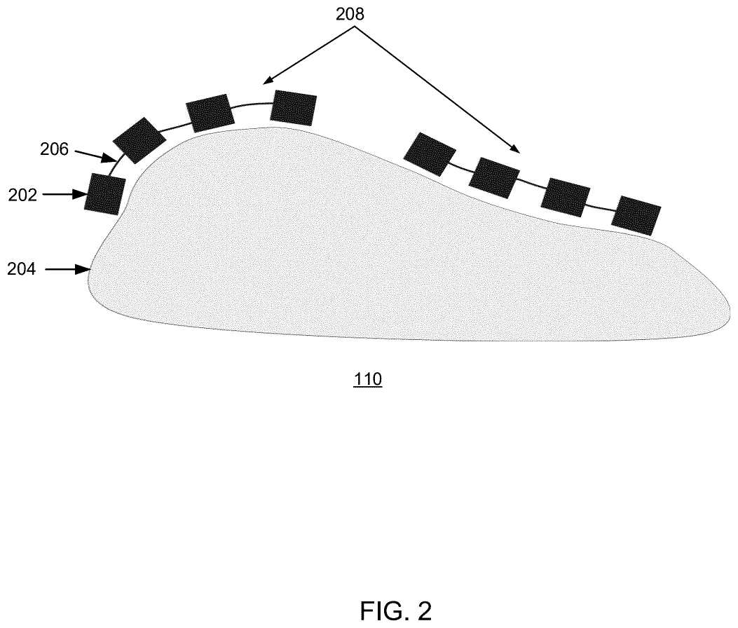

FIG. 2 illustrates an example arrangement and configuration of a flexible ultrasound transducer array 110 employed in the system 100, according to some embodiments of the disclosure. The array 110 includes multiple transducer elements 202, strategically positioned to maximize acoustic coupling with the target object, denoted as 204. These transducer elements 202 may be fabricated from piezoelectric materials such as lead zirconate titanate (PZT), polyvinylidene fluoride (PVDF), or other flexible piezoelectric polymers that are capable of both generating and receiving ultrasound waves. It should be noted, while flexible transducer arrays are illustrated, the disclosed method and system are not limited to. For example, the disclosed method and system may be also applied to fixed or rigid transducer arrays.

The transducer elements 202 may be arranged in a matrix configuration and are embedded within or attached to a flexible substrate, labeled as 206. The substrate 206 may be composed of biocompatible, stretchable materials such as silicone rubber, thermoplastic polyurethane (TPU), or other elastomeric compounds. This composition allows the substrate to deform without compromising the functional integrity of the transducer elements 202, enabling the array 110 to conform to complex surface geometries, such as curved or irregular anatomical structures, as shown in FIG. 2.

In some embodiments, the substrate 204 may be configured with segmented sections, allowing specific transducer elements to move independently, as indicated by two arrows 208. This modular configuration provides greater spatial flexibility, enabling the array to maintain optimal contact with non-planar surfaces or dynamically moving objects. Additionally, the segmented design may mitigate potential acoustic interference between adjacent elements, thereby enhancing the clarity and spatial resolution of acquired ultrasound data.

In some embodiments, the transducer elements 202 themselves may be configured to operate in both transmit and receive modes, allowing for comprehensive data acquisition from various angles and orientations. Each element may be equipped with an individual signal control line, enabling independent actuation and data collection. The element spacing, defined by the inter-element pitch, may be optimized to minimize spatial aliasing while maintaining a high spatial resolution. The typical pitch distance ranges from 1 mm to 2 mm, depending on the operating frequency and desired imaging depth.

In some embodiments, the flexible substrate 204 may include integrated electrical interconnections to facilitate data transmission from each transducer element to the ultrasound data acquisition module 122. These interconnections may be designed to withstand repeated stretching and deformation without signal degradation, ensuring consistent data integrity throughout the imaging process.

The structure shown in FIG. 2 also illustrates potential methods of attachment to the target object. The array 110 may be affixed using adhesives, straps, or mechanical clamps, depending on the application requirements. In biomedical applications, biocompatible gel or coupling agents may be applied between the array 110 and the skin surface to enhance acoustic coupling and reduce signal reflection.

The architecture depicted in FIG. 2 underscores the adaptability of the transducer array 110 to varied surface geometries and dynamic environments. By utilizing flexible materials and modular configurations, the system 100 may achieve precise spatial mapping while maintaining consistent acoustic coupling, thus enabling accurate shape estimation and high-resolution imaging in both medical and industrial contexts.

### Ultrasound Data Acquisition Module

The ultrasound data acquisition module 122 may be configured to capture, digitize, and temporarily store raw ultrasound data from the transducer elements, ensuring that the data is accurately represented and synchronized for subsequent signal preprocessing and feature extraction. This module is essential for maintaining data integrity and optimizing the overall imaging and shape estimation process.

In some embodiments, the data acquisition module 122 may initiate the imaging or shape estimation process by transmitting electrical signals to the transducer elements. These electrical pulses may be converted into ultrasound waves through the piezoelectric effect. The transducer elements then emit these ultrasound pulses (also referred to as ultrasound waves) toward the target object, which may be biological tissue, industrial components, or other scanned surfaces. After transmitting the ultrasound pulses, the transducer elements may switch to the receive mode to detect reflected echoes. These echoes are mechanical vibrations resulting from the interaction of ultrasound waves with structures within the target object. The transducer elements convert these mechanical vibrations back into electrical signals, effectively functioning as receivers. The received signals contain critical information regarding the internal structure of the target object, including ToF, amplitude, and phase data. This data provides spatial and temporal information that is essential for determining the relative positions of transducer elements and for reconstructing the target's internal geometry.

In some embodiments, the transmission of ultrasound waves from the transducer elements may be executed in various modes. The transmission may occur individually from each element, concurrently from multiple elements, or in a specific sequence to achieve desired spatial coverage or imaging effects. Additionally, individual time delays may be applied to the transmission of each element to steer or focus the ultrasound waves, thereby directing the acoustic energy towards specific regions of interest. This approach allows for more precise targeting and may enhance imaging resolution or facilitate the examination of complex structures.

In some embodiments, the transmitted pulse may take different forms, including a single-frequency wave or a frequency-modulated “chirp.” A single-frequency pulse provides targeted, narrowband energy, whereas a chirp involves a sweep across multiple frequencies, potentially improving penetration depth and signal-to-noise ratio. After transmission, selected transducer elements switch to receive mode, capturing the returning echoes. These echoes include direct signals from the transmitting element to the receiving element as well as reflections from inhomogeneities within the object under inspection. The received signals provide valuable information about the internal structure, enabling subsequent analysis to identify spatial configurations or detect structural abnormalities.

In some embodiments, following the reception, the received analog signals may be routed to the analog-to-digital converter (ADC), a vital component that digitizes the analog signals at a predefined sampling rate (also referred to as sampling frequency). The sampling rate is selected based on the desired resolution and operating frequency range of the transducer array. In some embodiments, the data acquisition module 122 may further implement a data buffering system to temporarily store the acquired digital signals. This step is crucial for maintaining data synchronization, particularly in multi-channel systems where multiple transducer elements are actively transmitting and receiving signals simultaneously. In some embodiments, the data acquisition module 122 may be configured to handle multi-channel data acquisition. In systems with large transducer arrays, each transducer element may operate as an independent data channel, simultaneously acquiring signal data. The data acquisition module 122 may incorporate multiplexers and signal routing circuits to manage multiple data streams and prevent data collisions. In some embodiments, the data acquisition module 122 may transmit the acquired digital data to other processing modules or store the data locally for subsequent processing.

In some embodiments, the acquired ultrasound data may be further subjected to signal preprocessing by a signal preprocessing module (not shown in FIG. 1, but may be a part of the ultrasound data acquisition module 122 or the feature engineering module 124, depending on the configuration of the system 100). Exemplary signal preprocessing may include but is not limited to frequency filtering, time gain compensation, envelope extraction, signal segmentation, noise reduction, dynamic range compensation, etc. These signal preprocessing techniques are essential for enhancing signal quality and mitigating noise, thereby improving the accuracy of subsequent feature extraction.

Specifically, for frequency filtering, the signal preprocessing module may be configured to isolate specific frequency bands relevant to the ultrasound transducer's operating range. Ultrasound signals generally comprise multiple frequency components, some of which may carry noise or irrelevant data that may obscure key features such as first arrivals or reflective echoes. By applying bandpass filtering, the system may suppress unwanted frequency components while retaining the frequencies that contain pertinent spatial information. For instance, in applications where the transducers operate in the 1 MHz to 20 MHz range, the filter is configured to pass only signals within this range, thereby reducing background noise and improving signal integrity.

With respect to the time gain compensation, the signal preprocessing module may be configured to address the natural attenuation of ultrasound signals as they propagate through tissue or other media. As sound waves travel further from the transducer, their amplitude decreases, potentially leading to weaker echoes from deeper regions. Time gain compensation may compensate for this loss by selectively amplifying the received signals based on their depth or travel time. This adjustment is crucial for maintaining consistent amplitude levels across different spatial regions, ensuring that deeper echoes remain detectable and informative.

With respect to the envelope extraction, raw ultrasound signals generally exhibit oscillatory waveforms, making it challenging to directly assess amplitude profiles and spatial patterns. Envelope extraction may isolate the amplitude profile by converting the oscillatory signal into a smooth, continuous waveform that represents the signal's overall intensity. This process involves demodulating the raw signal to identify key amplitude peaks and suppress oscillatory components, thereby generating a signal envelope that is easier to interpret during feature extraction.

With respect to the dynamic range compensation, it is a technique designed to standardize the amplitude range of the received signals. Raw ultrasound signals often exhibit a wide amplitude range, with strong reflections from dense structures and weaker echoes from softer tissues. Dynamic range compression may reduce the disparity between high and low amplitude signals, ensuring that subtle echoes remain discernible without overwhelming the primary signal components. This step may be achieved using logarithmic scaling or other amplitude normalization techniques.

With respect to noise reduction, the signal preprocessing module may be configured to suppress signal artifacts and spurious echoes. Common noise reduction techniques include median filtering, which eliminates isolated noise spikes, and adaptive filtering, which adjusts the filtering parameters based on signal characteristics. These techniques are particularly important in applications involving flexible arrays, where transducer deformation may introduce mechanical noise or electrical interference.

With respect to signal segmentation, the signal preprocessing module may be configured to isolate specific regions of interest (ROIs) within the ultrasound data. This segmentation process may involve identifying key signal components, such as the first arrival wavefront or specific echo patterns, which are particularly relevant for spatial estimation. By segmenting the signal data into distinct ROIs, the system may focus its analysis on critical spatial features, reducing computational complexity and enhancing the accuracy of subsequent shape estimation.

In some embodiments, focused imaging data may be processed to derive the full multistatic dataset. In this context, focused imaging refers to the acquisition of data using beamforming techniques that concentrate acoustic energy on specific regions or focal points. The resulting focused data may then be processed to simulate a multistatic dataset, where each transducer element acts as both a transmitter and a receiver in different configurations. This approach may effectively emulate FMC data acquisition, generating comprehensive datasets that include all possible transmit-receive pairs. The derived multistatic dataset may provide a rich source of spatial and temporal information, enabling more accurate spatial mapping, enhanced resolution, and improved imaging clarity.

### Feature Engineering Module

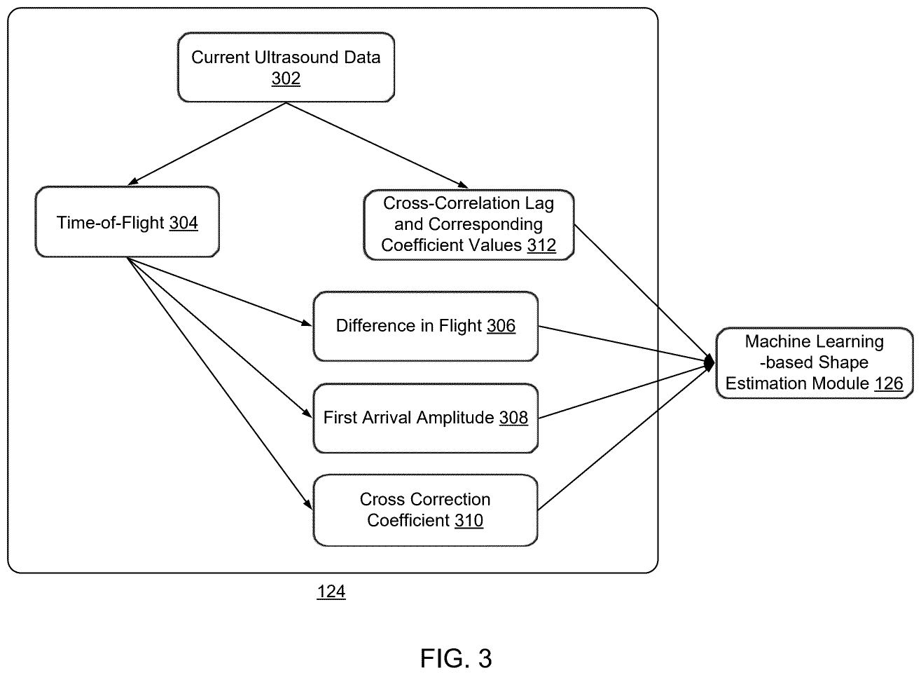

FIG. 3 illustrates an example feature extraction process implemented by the feature engineering module, according to some embodiments of the disclosure. This process is critical for accurately estimating the spatial configuration of transducer elements based solely on the acquired ultrasound data. The diagram outlines the sequence of operations involved in extracting key spatial features, which are subsequently utilized as inputs to a machine learning model for shape estimation.

Following the aforementioned signal preprocessing, the feature engineering module 124 may extract a set of key features from the ultrasound data. These features may be categorized into several distinct types. The first set of features pertains to the ToF 304 of transmitted acoustic wavefronts between transducer elements. ToF is a fundamental parameter in ultrasound imaging as it provides direct distance measurements based on the travel time of ultrasound pulses. To calculate ToF, the system 100 may monitor the time taken for the ultrasound wave to propagate from a transmitting element to a receiving element. This may be achieved through methods such as peak amplitude detection, where the first significant amplitude peak in the received signal is identified, or through cross-correlation of the first arriving ultrasound signals. By comparing the ToF values between multiple transducer pairs, the system 100 may derive spatial relationships and relative distances between elements, forming the foundation for subsequent shape estimation.

In addition to ToF 304, another critical feature extracted by the feature engineering module 124 relates to the difference in flight (or difference in arrival time) 306 between neighboring elements. This parameter is particularly useful in detecting small spatial displacements or deformations within the transducer array. By analyzing the time difference between the first arrivals of ultrasound pulses at adjacent transducer elements, the system may identify local shifts in spatial configuration. This information is especially valuable in scenarios involving dynamic surfaces or non-planar geometries, where the array may undergo slight positional changes during data acquisition.

Another essential feature extracted by the feature engineering module 124 during this phase is the first arrival amplitude 308. This amplitude represents the intensity of the initial ultrasound echo received by each transducer element. Since amplitude is influenced by factors such as surface reflectivity and tissue density, the first arrival amplitude 308 may provide additional spatial context that complements the ToF data 304. For instance, variations in amplitude across the transducer array may indicate changes in material properties or surface irregularities. By integrating amplitude data with ToF measurements, the system 100 may achieve a more comprehensive representation of the spatial configuration.

A further feature extracted by the feature engineering module 124 may relate to the cross-correlation coefficient 310 calculated from the first arriving ultrasound signals received by different transducer element pairs. Cross-correlation coefficient measures the similarity between two waveforms and helps to identify spatial relationships between elements based on the degree of signal alignment. In this context, the system 100 may compute the cross-correlation coefficients between the first arrival signals of neighboring transducer elements. Higher correlation values indicate closer spatial alignment, while lower values may suggest greater separation or deformation. This information is particularly useful for refining spatial estimates and detecting subtle positional changes within the transducer array.

Additionally, the feature engineering module 124 may also extract features related to the entire ultrasound channel data, as opposed to only the first arriving wavefront. In this case, the cross-correlation lag and corresponding coefficient values 312 may be calculated for the full set of received signals across all transducer pairs. This approach may enable the system 100 to capture more extensive temporal relationships and signal patterns that may not be evident from the first arrival data alone. By analyzing the entire signal sequence, the system 100 may detect secondary echoes, multi-path reflections, and other temporal features that contribute to more accurate spatial mapping.

In some embodiments, the engineered features 304-312 obtained by the feature engineering module 124 from the current ultrasound data 302 may be input into the DNN or other machine learning models included in the machine learning-based shape estimation module 126 for shape estimation, as will be described in detail later. By inputting derived features into the DNN instead of directly feeding the raw ultrasound RF data, the generalizability and accuracy of the model may be significantly improved. Feature engineering or feature extraction thus serves as a targeted preprocessing step that extracts specific spatial and temporal attributes from the RF data, effectively providing the machine learning model with task-specific inputs that are more relevant to the objective of element localization.

This approach is analogous to offering the DNN a structured, task-oriented starting point that encapsulates essential information such as first arrival time, amplitude, and cross-correlation coefficients. These engineered features act as focused indicators of spatial relationships and positional variations within the transducer array, allowing the model to discern underlying spatial patterns more effectively. As a result, the model may more readily identify and learn spatial dependencies, reducing the overall complexity of the localization problem. Moreover, the engineered features are derived from the fundamental physics of ultrasound propagation and the structural characteristics of the same object being imaged, making them uniquely tailored to the specific task of spatial configuration estimation. This physics-based approach distinguishes the derived features from conventional deep learning features, which may not inherently capture spatial or temporal nuances essential for accurate localization. Thus, the integration of engineered features not only simplifies the learning process but also enhances the model's ability to generalize across varied transducer configurations and target surfaces.

In some embodiments, before the extracted features are input into the machine learning model in the module 126, these features may be further subjected to a feature preprocessing process, which may be different from the aforementioned signal preprocessing, and may be focused on structuring, organizing, and refining the extracted features derived from the preprocessed signals. The feature preprocessing may be implemented through a feature preprocessing module (not shown in FIG. 1, but may be a part of the feature engineering module 124 or the machine learning-based shape estimation module 126, depending on the configuration of the system 100). The goal here is to convert the extracted features into a structured feature set that may be effectively used as input for the machine learning model. Exemplary feature preprocessing may include but is not limited to data structuring (e.g., organizing the extracted features into a consistent format, such as vectors, matrices, or tensors, to ensure compatibility with the neural network), normalization (e.g., scaling features to a consistent range (e.g., between 0 and 1) to prevent certain features from dominating the learning process), dimensionality reduction (e.g., applying techniques like principal component analysis (PCA) or linear discriminant analysis (LDA) to reduce feature dimensionality, thus minimizing computational load and preventing overfitting), feature selection (e.g., identifying and selecting the most relevant features, such as ToF, first arrival amplitude, and cross-correlation coefficients, to focus on the most informative data while discarding redundant or irrelevant features), encoding (e.g., transforming complex spatial and temporal relationships into formats that the DNN can readily process, such as embedding spatial relationships as relative distance matrices or adjacency matrices), etc. The output of feature preprocessing is thus a structured feature set ready to be inputted into the deep neural network for spatial coordinate estimation. Unlike the output of signal preprocessing, which remains in the form of processed ultrasound signals, the output the feature preprocessing is a refined dataset comprising numerical features that encapsulate spatial, temporal, and amplitude information.

In some embodiments, the extracted various features may be combined to form the input dataset for the machine learning model. This step is critical for ensuring that the data is organized in a format that optimizes the learning process and enables the model to accurately infer spatial configurations of the transducer elements. The combination of features may be performed in various ways. In one approach, the features may be combined linearly, where each feature is sequentially ordered or interleaved to form a one-dimensional input vector. This structure may maintain the temporal or spatial sequence of the features, allowing the model to learn patterns based on signal progression or positional relationships. Alternatively, the features may be arranged as a matrix as described above, wherein each row or column represents a specific feature (e.g., first arrival time, amplitude, cross-correlation), and each entry corresponds to a specific transmit-receive pair. This matrix format may enable the model to capture spatial dependencies more effectively, as it preserves the positional relationships between transducer elements. In another configuration, the inputs may be separated and arranged by transmit or receive events. For example, all features associated with a specific transmitting element may be grouped together, followed by the features associated with the next transmitter. This organization may be particularly useful in scenarios where the transmit-receive configuration varies dynamically, such as in FMC or synthetic aperture imaging. By structuring the input data in these specific formats, the machine learning model may better learn spatial relationships and temporal patterns, ultimately improving its ability to accurately estimate the spatial configuration of the transducer array.

### Machine Learning-Based Shape Estimation Module

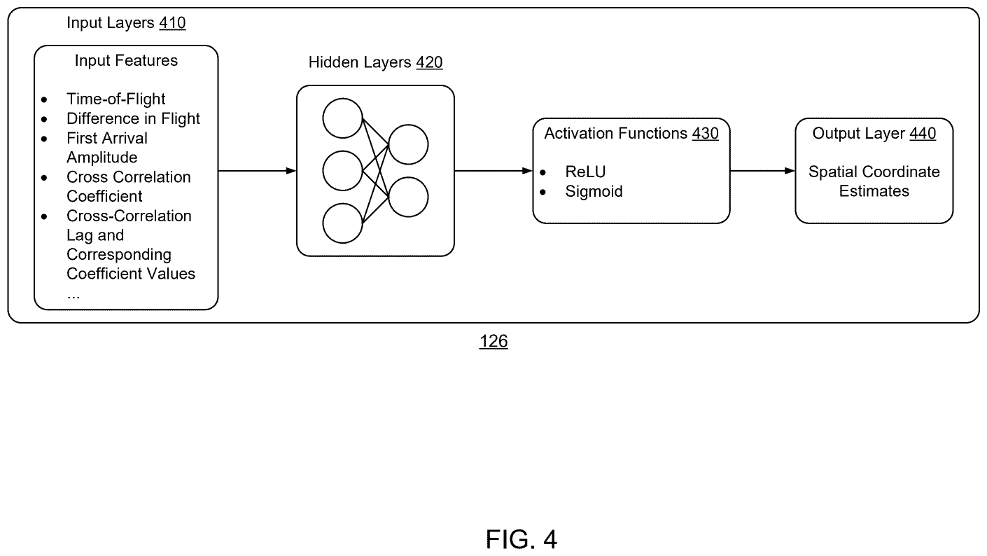

Various machine learning models may be utilized for shape estimation based on the extracted features described above. Besides DNN described earlier, convolutional neural network (CNN) is another machine learning model that may be utilized to extract spatial patterns from matrix-structured inputs, such as feature maps representing transmit-receive configurations. By applying convolutional filters, the CNN may effectively detect localized spatial features, such as alignment patterns or positional clusters, making it particularly suitable for scenarios involving complex or irregular surface geometries. Additionally, other network architectures, such as recurrent neural networks (RNNs) or long short-term memory (LSTM) networks, may be also implemented to capture temporal dependencies in the data. These networks are particularly useful in applications where the transducer configuration may change dynamically over time, as they can maintain memory of previous spatial states and adjust predictions based on sequential inputs. In the following, the DNN is used as an example architecture for illustrative purposes, as shown in FIG. 4.

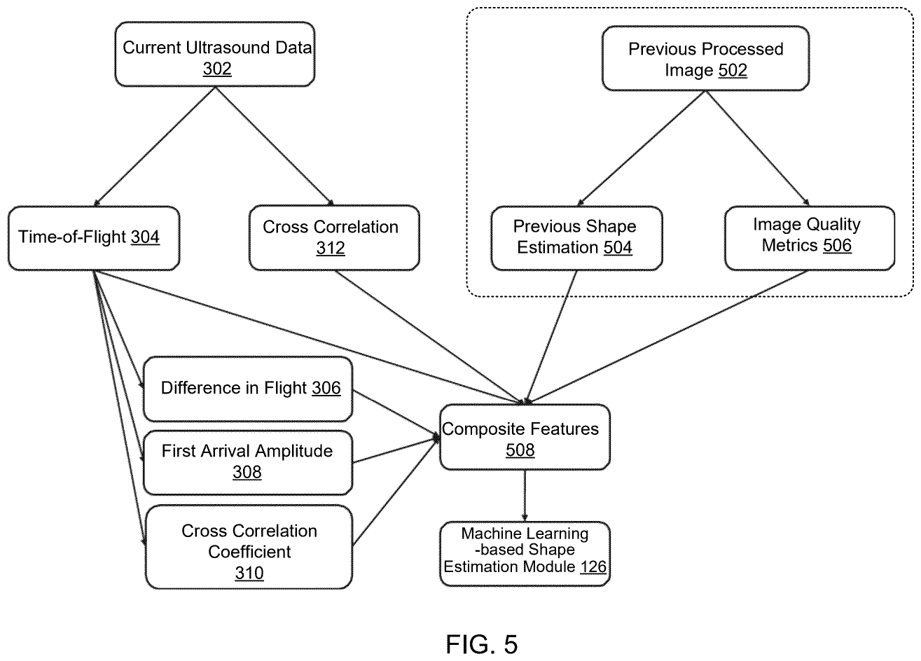

The input data into the machine learning model may include a comprehensive feature set, including ToF 304, difference in flight 306, first arrival amplitude 308, cross-correlation coefficient 310, and cross-correlation lag and corresponding coefficient values 312, and so on as shown in FIG. 4. Alternatively, a composite feature set 508 in FIG. 5 may be input into the machine learning model. Each feature provides specific spatial and/or temporal information that aids in determining relative distances and positional relationships between transducer elements.

The DNN architecture may be configured to capture both local and global spatial relationships within the feature set. The network may include multiple layers, each serving a distinct purpose in the spatial estimation process. For example, the input layer 410 may be configured to receive the preprocessed feature set. Each input node may represent a specific feature, such as ToF data from a specific transmit-receive pair or amplitude data from a particular transducer element. The hidden layers 420 may be composed of multiple neurons interconnected through weighted links. These layers may be configured to identify complex spatial patterns and correlations across the feature set. In some embodiments, the hidden layers 420 may include optional convolutional layers and fully connected layers. The convolutional layers may be configured to detect local spatial patterns and feature interactions. This is particularly useful for identifying regional correlations between neighboring transducer elements. The fully connected layers may be configured to aggregate information from all preceding layers, integrating local and global spatial features to form a comprehensive spatial representation. The activation functions 430 include non-linear activation functions, such as rectified linear unit (ReLU), sigmoid or the like, which are employed to introduce non-linearity into the model, enabling the network to learn complex spatial dependencies and non-linear relationships between features. The output layer 440 may be configured to generate the spatial coordinate estimates for each transducer element. Depending on the dimensionality of the required spatial configuration, the output may consist of 2D coordinates (x, y) or 3D coordinates (x, y, z) for each transducer clement.

In some embodiments, before the deployment, the DNN may be trained to learn to map the input feature set to the spatial coordinates through supervised learning (or through unsupervised training). The training dataset includes pairs of feature sets and corresponding ground truth coordinates, and may include synthetic and real ultrasound data. The learning objective is to minimize the error between the predicted coordinates and the actual coordinates by adjusting the weights and biases of the network. In some embodiments, a suitable loss function may be selected to quantify the discrepancy between the predicted coordinates and ground truth coordinates. Exemplary loss functions that may be used herein include but are not limited to mean squared error (MSE): computes the average squared difference between predicted and actual coordinates, emphasizing larger errors; mean absolute error (MAE): measures the average absolute difference, providing a more robust measure against outliers; Huber loss: combines the advantages of MSE and MAE by being less sensitive to outliers while penalizing large errors. In some embodiments, the loss function is minimized using optimization algorithms such as stochastic gradient descent (SGD), Adam, or RMSprop. These algorithms may iteratively update the network weights to reduce the loss and improve coordinate estimation accuracy. In some embodiments, during each training iteration, the error may be propagated backward through the network using the backpropagation algorithm. This process may adjust the weights and biases to optimize the network's spatial inference capabilities.

Once the DNN is trained, it is capable of inferring spatial coordinates in real-time based on newly acquired ultrasound data. During the inference phase, the network may process the input feature set through the same series of layers and activation functions, generating spatial coordinate estimates as output. For example, the trained DNN may effectively learn the spatial relationships embedded within the feature set, allowing it to infer the relative positions of transducer elements based on ToF, amplitude, correlation data, etc. The output layer 440 then provides a set of spatial coordinates for each transducer element. These coordinates are represented as 2D or 3D vectors, depending on the desired spatial configuration.

In some embodiments, to further refine the output coordinates, the network may incorporate post-processing steps that leverage historical data or known spatial constraints. For instance, the predicted coordinates can be cross-referenced with previous frames to ensure temporal consistency and detect potential anomalies in the spatial configuration. In other words, beyond the current frame data, the system may leverage information derived from previous ultrasound image frames and shape estimations, as shown by block 504 in FIG. 5. This historical data 504 serves as a reference for detecting temporal changes in transducer positions, allowing the system to track spatial configurations over time. For instance, the localization result or coordinate estimates from a previously processed image 502 may be integrated into the input feature (e.g., as a part of the composite features 508 as shown in FIG. 5) to provide temporal context to predict the current spatial configuration. This predictive approach is particularly beneficial in applications involving dynamic or moving surfaces, where the transducer array may undergo continuous movement or deformation and thus the transducer positions may shift over time.

In some embodiments, the system 100 may further incorporate image quality metrics 506 derived from previous image frames as additional input features (e.g., as a part of the composite features 508 shown in FIG. 5). The quality metrics may include but are not limited to entropy, sharpness, brightness, and coherence, which together or individually may provide quantitative assessments of image quality, which may be further utilized by the machine learning-based shape estimation module 126 to refine the spatial estimates generated by the DNN. Unlike traditional iterative optimization methods, which rely on minimizing or maximizing specific image quality metrics, the disclosed system 100 may integrate these metrics as feature inputs without performing iterative optimization. This approach not only reduces computational complexity but also enables the machine learning model to learn complex spatial relationships directly from the input data.

In some embodiments, the leveraged information derived from previous ultrasound image frames and shape estimations and image quality metrics derived from previous image frames may be combined with the extracted features from the current ultrasound data to form a set of composite features (e.g., composite features 508 in FIG. 5), which are together input into the machine learning model for spatial estimation, as described earlier. The combination of composite features may be performed in various ways, such as combined linearly, arranged as a matrix, separated and arranged by transmit or receive events, as also described earlier.

In some embodiments, the trained DNN may also be capable of dynamically adapting its coordinate estimates based on new input data. In applications involving dynamic surfaces or moving objects, the transducer array may undergo positional shifts over time. The DNN may thus continuously update the spatial coordinates by reprocessing the same feature set, updated data in real-time, effectively accommodating changes in surface geometry or transducer alignment. This includes reprocessing the feature set including the composite features 508 in FIG. 5. By incorporating feedback from previous frames or incorporating image quality metrics, the DNN may refine its spatial estimates, ensuring that the generated coordinates remain accurate and consistent throughout the imaging sequence.

In some embodiments, the machine learning-based shape estimation module 126 may be further configured for spatial mapping and shape estimation by utilizing the spatial coordinates of the transducer elements as estimated by the DNN. These coordinates represent the relative positions of the transducer elements in 2D or 3D space, forming the spatial configuration map of the array. Specifically, the spatial mapping process may include translating these coordinates into a structured grid or matrix that represents the spatial layout of the transducer array. This grid may serve as the basis for aligning the ultrasound data and defining the relative positions of each transducer element. The mapped coordinates may then be integrated with the corresponding ultrasound data, establishing a spatial reference frame that is essential for accurate image reconstruction by the imaging module 128. In some embodiments, the machine learning-based shape estimation module 126 may implement error correction techniques to refine the spatial estimates. For instance, temporal filtering or smoothing algorithms may be applied to mitigate abrupt positional changes or anomalies in the coordinate data, ensuring that the spatial map is consistent over consecutive frames.

### Imaging Module

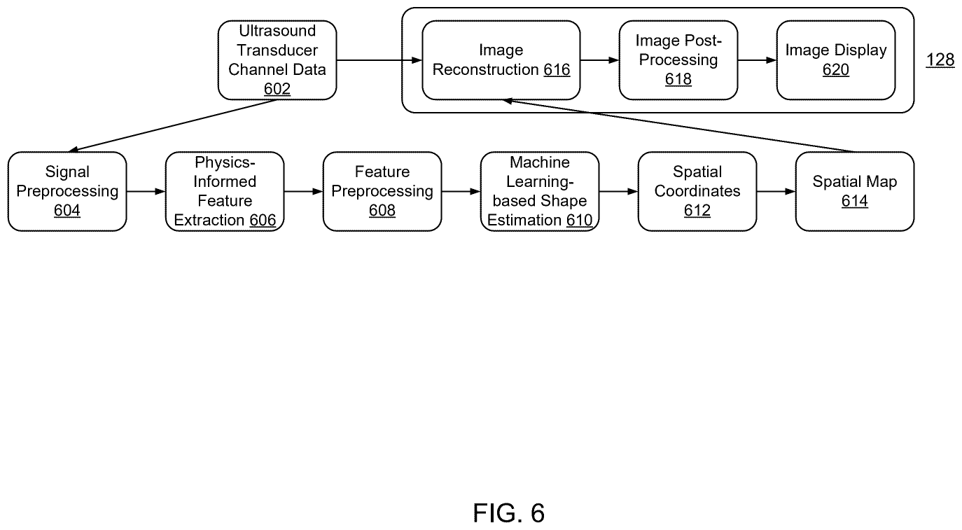

The imaging module 128 in the disclosed system 100 serves as the final stage of the ultrasound data processing pipeline as shown in FIG. 6, where the spatial configuration estimates and acquired ultrasound data are utilized to reconstruct high-resolution images of the target object. As described above and as shown in FIG. 6, the spatial configuration estimation may include signal preprocessing 604 of ultrasound transducer channel data 602, physics-informed feature extraction 606, feature preprocessing 608, machine learning-based shape estimation 610 to obtain the spatial coordinates 612 and further the spatial map 614 of the transducer elements. The estimated shapes of the transducer elements then establish a spatial reference frame that is essential for accurate image reconstruction by the imaging module 128, which itself is responsible for generating, refining, and displaying the ultrasound image, incorporating various algorithms and post-processing techniques to ensure accurate visualization of the object under the examination.

Image reconstruction 616 is the core function of the imaging module 128. It involves processing the ultrasound data in conjunction with the spatial map to generate a coherent visual representation of the target object. The reconstruction process may be executed using various algorithms, each suited to different imaging conditions and spatial configurations.

According to one embodiment, DAS may be utilized in the image reconstruction. DAS is a beamforming algorithm used in ultrasound imaging. In this method, the acquired ultrasound signals may be time-aligned based on the calculated ToF values for each transducer clement. The ToF values may be derived from the spatial configuration map, allowing the system to align signals based on the relative distances between transmit and receive elements. For each pixel in the reconstructed image, the algorithm may sum the time-aligned signals to enhance the signal strength and reduce noise. The resulting summed signal amplitude is assigned to the corresponding pixel location, forming the image. The DAS algorithm is computationally efficient but may exhibit artifacts in heterogeneous media due to phase mismatches and signal interference (e.g., off-axis scattering, reverberation, etc.).

According to another embodiment, modified DMAS (MDMAS) may be utilized in the image reconstruction. MDMAS is an enhancement of the standard DAS algorithm, incorporating amplitude weighting and adaptive filtering to further refine the image quality. In MDMAS, the signals may be weighted based on amplitude, coherence, or other quality metrics before summing. This weighting process may facilitate the reduction of side lobes and noise suppression, resulting in improved contrast and spatial resolution. MDMAS is particularly useful in scenarios involving flexible or deformable arrays, where transducer elements may shift, causing misalignment and phase differences. By incorporating amplitude weighting, MDMAS may mitigate these issues, producing more consistent and artifact-free images.

According to another embodiment, coherence-based imaging may be utilized in image reconstruction. For applications involving complex or heterogeneous media, the imaging module may employ coherence-based beamforming techniques, such as delay-multiply-and-sum (DMAS) or synthetic aperture focusing techniques (SAFT). With respect to DMAS, this algorithm may multiply the aligned signals before summing, effectively emphasizing coherent signal components and suppressing incoherent noise. DMAS is thus well-suited for applications with high noise levels or multi-path reflections, as it enhances signal coherence. With respect to SAFT, this technique may synthesize a larger aperture by combining multiple transducer channels, effectively increasing the spatial resolution and depth of field. SAFT may also compensate for transducer element displacement, making it ideal for flexible arrays.

In some embodiments, besides DAS, MDMAS, DMAS, SAFT techniques described above, other image reconstruction techniques may also be utilized by the imaging module 128. For example, adaptive beamforming techniques, such as minimum variance distortionless response (MVDR) beamforming, linearly constrained minimum variance (LCMV) beamforming, and eigenvalue-based beamforming may be utilized here. For another example, Fourier domain beamforming (FDB) may be utilized, where the received signals are processed in the frequency domain rather than the time domain. For another example, a total focusing method (TFM) may be utilized, where images are reconstructed by focusing on every pixel in the image plane using all possible transmit-receive pairs. For another example, compressive sensing (CS) may be utilized, which leverages signal sparsity to reconstruct images from fewer data samples than conventional methods, reducing data acquisition time and storage requirements. According to some embodiments, neural network-based techniques may also be utilized in the image reconstruction here. Deep learning models, particularly CNNs, have shown significant promise in ultrasound image reconstruction. In some embodiments, hybrid techniques may be utilized in the image reconstruction, which means multiple algorithms may be combined to leverage their respective strengths in image reconstruction. For example, DAS may be combined with DMAS to balance computational efficiency and resolution, CNN-based reconstruction may be integrated with coherence-based metrics for adaptive imaging in heterogeneous media, TFM may be combined with compressive sensing to achieve high-resolution imaging with reduced data acquisition, etc.

The choice of reconstruction technique depends on several factors, including the target medium, computational resources, desired spatial resolution, and data acquisition strategy. While techniques like DAS and MDMAS provide a baseline for real-time imaging, advanced methods such as SAFT, adaptive beamforming, DMAS, and TFM offer superior spatial resolution and contrast, albeit at a higher computational cost. Emerging methods such as compressive sensing and neural network-based reconstruction promise significant advancements in image quality and processing speed, particularly in dynamic or complex imaging scenarios.

Referring back to FIG. 6, in some embodiments, the imaging module 128 may perform certain post-processing 618 to enhance image quality and clarity. Example post-processing techniques may include but are not limited to log compression, envelope detection, noise reduction and smoothing, dynamic range adjustment, etc. Specifically, for log compression, the amplitude data may be converted into a logarithmic scale, reducing the dynamic range and enhancing subtle contrast variations. This technique is particularly effective for visualizing soft tissue structures. For envelope detection, the amplitude envelope of the reconstructed signal may be extracted, smoothing oscillations and producing a continuous intensity profile for each pixel. This step is crucial for creating visually coherent images. For noise reduction and smoothing, filters such as median filtering, Gaussian smoothing, or adaptive filters may be utilized to suppress residual noise and artifacts. These filters are configured to preserve edge information while reducing speckle and other high-frequency noise components. For dynamic range adjustment, the intensity range of the image may be adjusted to optimize contrast, ensuring that both strong and weak echoes are visible. This step prevents signal saturation and enhances the visibility of low-amplitude echoes.

In some embodiments, post-image processing extends beyond visual enhancement and includes the analysis of both quantitative and qualitative information derived from the ultrasound data. This advanced processing is aimed at extracting functional and biomechanical properties of the tissue or material being imaged, providing clinically or operationally relevant insights beyond conventional B-mode imaging. One such application is elastography, a technique used to measure tissue stiffness by evaluating how tissues deform in response to mechanical stress. Elastography may help detect abnormalities such as tumors or fibrosis, which typically exhibit increased stiffness compared to surrounding healthy tissue. By processing the spatially estimated and reconstructed ultrasound data, the system may generate elasticity maps that overlay mechanical property information onto anatomical images. Another important post-processing feature is sound speed mapping, which involves calculating the local speed of sound within different regions of the object. Variations in sound speed are indicative of differences in tissue composition, density, or pathology. This information may enhance the accuracy of spatial localization and image reconstruction, particularly in heterogeneous or layered media. Additionally, blood flow analysis, often achieved through Doppler processing, may be used to visualize and quantify the movement of blood within vessels. This includes parameters such as flow velocity, direction, and volume, which are critical for assessing vascular health and function. By integrating blood flow metrics with structural imaging, the system may support more comprehensive diagnostic assessments, such as detecting blockages or assessing perfusion. Additional quantitative and qualitative not described above are also possible and contemplated by the disclosure. These extended post-processing capabilities enrich the imaging output by delivering functional diagnostics alongside anatomical insights, enabling applications in fields such as oncology, cardiology, musculoskeletal imaging, and real-time physiological monitoring.

In some embodiments, the imaging module 128 may be configured to process data continuously, allowing for dynamic visualization of the target object. To achieve this, the module 128 may implement adaptive control mechanisms that adjust acquisition parameters based on the estimated transducer configuration. For instance, if the spatial map estimated by the machine learning-based shape estimation module 126 indicates a significant displacement of transducer elements, the module 128 may adjust the beamforming parameters to compensate for the positional shift. This adaptation may ensure that the reconstructed image remains accurate despite transducer deformation or movement.

In some embodiments, the imaging module 128 may incorporate feedback loops that assess image quality metrics, such as sharpness, entropy, and coherence. These metrics may be used to fine-tune acquisition parameters or optimize beamforming weights, further enhancing real-time imaging performance.

The final stage of the imaging module 128 involves rendering the processed image on a graphical user interface (GUI), as indicated by the image display 620 in FIG. 6. The GUI may provide visual feedback in the form of 2D or 3D ultrasound images, allowing operators to assess the target object's internal structure. In some implementations, the GUI may also display additional information, such as estimated transducer coordinates to verify array configuration, image quality metrics to assess the clarity and reliability of the reconstructed image, annotations or overlays highlighting specific regions of interest, such as tissue boundaries, lesions, or structural anomalies. In some embodiments, the GUI may further display a certain summary generated based on the ultrasound images. For example, for diagnostic purposes, the 2D or 3D ultrasound images generated from a user may provide information whether a target tissue or organ of the user is normal or abnormal. In some real applications, certain imaging tools and/or generative AI tools may further utilize the obtained 2D or 3D ultrasound images to generate a summary to indicate the presence or absence of a disease. In some embodiments, certain treatments may be further provided through a same or different GUI, including possible links directed to additional knowledge or information related to the disease or treatments of the identified disease.

### Example Implementation

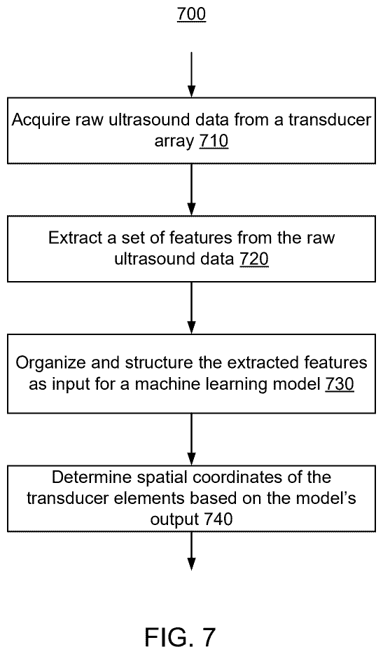

In the following, an example implementation of the disclosed machine learning-based shape estimation system 100 is further described. FIG. 7 is a flow chart of an example method 700 for machine learning-based shape estimation of transducer elements, according to some embodiments of the disclosure.

Step 710: Acquire raw ultrasound data from a transducer array.

The first step in the method 700 involves acquiring raw ultrasound data from the transducer array. This data includes the signals received by each transducer element after emitting ultrasound pulses and receiving echoes reflected by the target object. The transducer elements may be configured in a flexible or modular arrangement, allowing them to adapt to complex surface geometries. Since the spatial configuration of these elements is initially unknown, the objective is to derive positional information solely from the acquired signal data. The acquired data may include raw RF signals that include ToF information, amplitude profiles, and waveform characteristics. This dataset may form the basis for subsequent feature extraction, wherein specific spatial and temporal attributes may be identified to assist in determining the transducer configuration.

Step 720: Extract a set of features from the raw ultrasound data.

The next stage involves extracting meaningful features from the raw ultrasound data. These features may encapsulate critical spatial and temporal information necessary for spatial localization and shape estimation. The key features derived in this method may include first arrival times, difference between first arrival times of neighboring elements, cross-correlation coefficient, amplitude, total energy near detected first arrival time, and other additional features, such as phase information, envelope amplitude, and entropy, which may also be extracted to further enhance the spatial resolution and robustness of the localization algorithm.

Step 730: Organize and structure the extracted features as input for a machine learning model.

Once the relevant features have been extracted, these features may be organized and structured as input for a machine learning model. The specific process may refer to the feature preprocessing described earlier. For example, each feature (e.g., first arrival time, amplitude, cross-correlation) may be standardized and combined into a single input vector. This input vector represents a comprehensive spatial profile for each transducer element and serves as the primary input to the neural network. In addition, before being input to the machine learning model, the feature set may be normalized to ensure that all features contribute equally (or at a proper ratio) to the learning process, preventing any single feature (e.g., amplitude) from dominating the model.

Step 740: Determine spatial coordinates of the transducer elements based on the model's output.

The final stage involves inferring the spatial coordinates of the transducer elements based on the model's output. The choice of machine learning algorithm may vary, and in an example method, a deep neural network (DNN) is employed due to its capacity to model complex, non-linear spatial relationships. The DNN may generate a set of estimated coordinates that represent the spatial configuration of each transducer element in either 2D or 3D space. In some embodiments, the estimated coordinates by the machine learning model may be mapped to a structured grid or matrix, defining the relative positions of all transducer elements in the array. This spatial map may serve as a reference for subsequent imaging or structural analysis. In some embodiments, to ensure accuracy, the estimated positions are cross-referenced with previously acquired spatial data (if available) or validated using ground truth datasets. Additionally, the output may be refined using post-processing techniques, such as temporal filtering or adaptive correction, to mitigate errors and enhance localization accuracy.

In some embodiments, while not shown in FIG. 7, the method 700 may optionally include a step of using the determined spatial coordinates to align ultrasound data during beamforming and image reconstruction. By accurately mapping the transducer configuration, the system may ensure that the reconstructed image maintains spatial fidelity and provides a reliable representation of the scanned object, which then leads to potential applications in different fields.

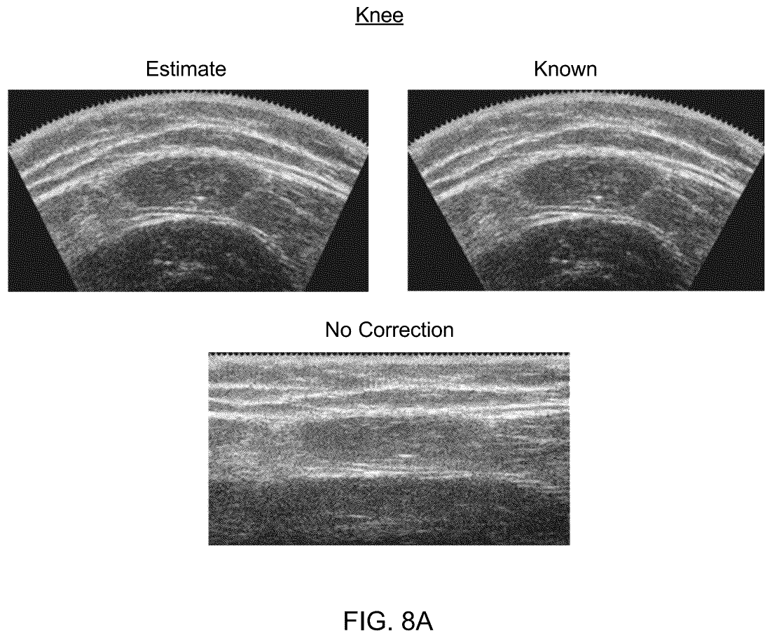

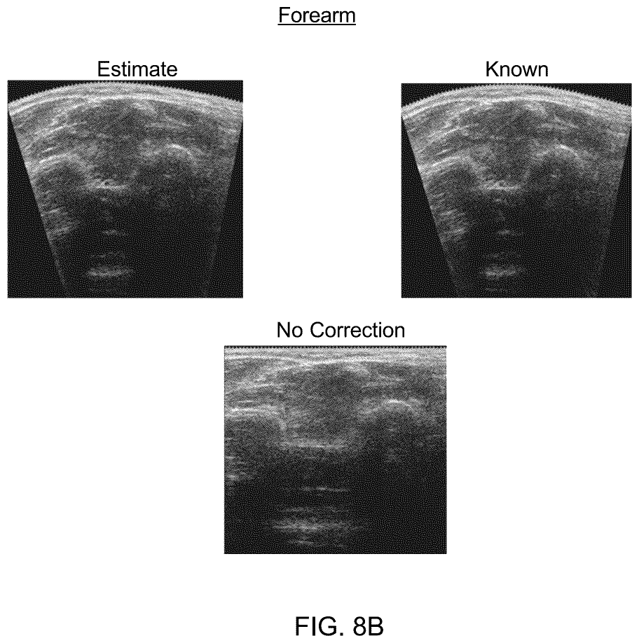

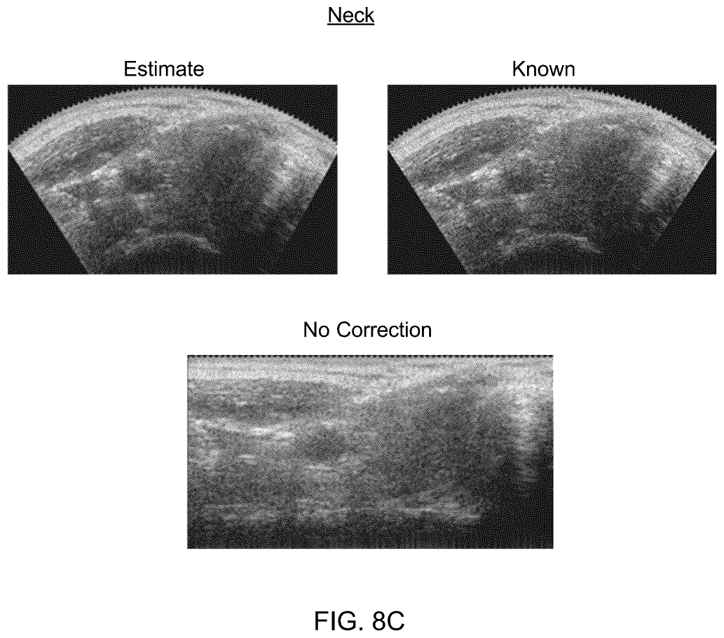

FIGS. 8A-8C illustrate the application and performance of the disclosed method 700 for estimating the spatial configuration of ultrasound transducer elements in three distinct anatomical regions: the knee, forearm, and neck. These figures demonstrate the system's ability to estimate the spatial arrangement of transducer elements without external correction or alignment, highlighting its effectiveness in dealing with complex, curved surfaces.

Specifically, FIG. 8A depicts a comparison of reconstructed imaging obtained based on the estimated transducer positions (“Estimate” in FIG. 8A) and the known reference configuration (“Known” in FIG. 8A), with “No Correction” serving as a negative control (i.e., a baseline imaging or localization scenario where the spatial configuration of the transducer elements is assumed to follow a standard, idealized shape, which may introduce errors and artifacts in the final image, especially when the array is flexed, stretched, or applied to non-planar surfaces). The known configuration represents the actual physical arrangement of the transducer elements as determined through a reference measurement, such as optical tracking or a fixed calibration setup or through other different means. The knee is a region characterized by a curved and irregular surface, making it a challenging area for accurate transducer localization. Despite these challenges, the system demonstrates a reasonable alignment between the estimated and known positions, indicating the model's capacity to accurately infer spatial configuration based solely on ultrasound data and extracted features. The method's robustness in handling surface curvature without correction demonstrates its applicability in wearable ultrasound systems or dynamic imaging scenarios.

FIG. 8B illustrates the estimation performance for a transducer array positioned on the forearm. The forearm presents a relatively less complex surface geometry compared to the knee, allowing for a more controlled evaluation of the model's spatial estimation accuracy. Similar to FIG. 8A, this figure compares reconstructed imaging obtained based on the estimated positions of the transducer elements with the known reference configuration. The data points indicate that the system achieves a high degree of spatial accuracy, with minimal deviation between the estimated and known coordinates. FIG. 8C shows the estimation results for the neck, a region characterized by complex surface curvature, muscular structures, and varying tissue densities.

Form the comparisons with the known reference configurations, FIGS. 8A-8C collectively illustrate the capability of the proposed method to estimate the spatial configuration of ultrasound transducer elements across anatomical regions with varying curvature and surface complexity.

### Example Applications

The enhanced imaging accuracy extends the capabilities of traditional ultrasound systems through the integration of deep learning and novel feature extraction methods disclosed herein. The potential applications may be categorized into medical imaging applications and non-medical industrial applications.

For medical imaging applications, the disclosed ultrasound imaging system may offer substantial improvements in terms of spatial resolution, imaging accuracy, and adaptability to dynamic surfaces. First of all, the disclosed ultrasound imaging system may be adapted to perform ultrasound CT, which differs from conventional ultrasound imaging by providing cross-sectional images similar to X-ray CT but without radiation exposure. This application leverages the technology's capability to accurately estimate the spatial configuration of transducer elements, enabling the reconstruction of high-resolution cross-sectional images that may assist in detecting abnormalities in soft tissues. Secondly, the flexible array design is particularly suitable for wearable ultrasound devices that may be applied to different body parts for continuous monitoring. Potential applications include tracking muscle motion for prosthetics or monitoring organ deformation in real time. The ability to maintain accurate spatial configuration during movement makes it valuable for such applications. Thirdly, the disclosed ultrasound imaging system may be adapted for therapeutic applications, including focused ultrasound for tumor ablation and blood clot dissolution and healing acceleration. For example, the accurate spatial mapping capabilities may enable precise targeting of focal regions, allowing the system to direct ultrasound energy to specific tissue areas to destroy tumors or other pathological structures. In addition, by focusing ultrasound waves at specific locations, the system may facilitate blood clot dissolution or promote tissue healing, potentially aiding in postoperative recovery or chronic wound treatment.

For industrial and non-medical applications, beyond medical imaging, the disclosed ultrasound imaging system also presents significant opportunities in industrial contexts, where it may be used for structural analysis, defect detection, and material characterization. Specific applications include structural integrity analysis, where the disclosed system may be employed to assess the integrity of industrial components, such as pipes, machinery, and aerospace structures. The ability to conform to complex surfaces allows for accurate mapping of structural defects, cracks, or corrosion in irregularly shaped objects. In addition, in robotic and automated inspection systems, the flexible ultrasound array may be integrated into robotic inspection systems to monitor the structural integrity of industrial assets in real time. By using deep learning-based spatial mapping, the system may dynamically adjust to surface contours, providing precise imaging even in challenging environments. Furthermore, the system's advanced feature extraction capabilities, including time-of-flight and amplitude analysis, may enable it to assess material properties such as density, elasticity, and porosity. This information is valuable for quality control in manufacturing processes or for evaluating material degradation over time.

In some embodiments, the disclosed system may provide additional insights for data-driven applications and simulations. For example, the disclosed system may utilize simulated ultrasound data under varying conditions for training, such as varying transducer spatial configurations, surface geometries, and acoustic properties of imaging media. This synthetic data may serve as a valuable training set for the deep learning model, improving its robustness and generalizability across diverse imaging scenarios. In some embodiments, in addition to the simulated synthetic data, the disclosed system may also utilize real-world datasets with known spatial configurations to verify and validate the model's performance. The real ultrasound data may include labeled datasets comprising ultrasound data acquired from predefined transducer configurations on different anatomical surfaces, including curved, irregular, and flat surfaces. This capability is particularly important in regulated medical environments, where accurate spatial mapping and localization are essential for clinical safety and efficacy. The ability to work with both simulated and real-world data also enhances the system's adaptability and scalability, making it suitable for commercial deployment in diverse medical and industrial applications.

### Operating Apparatus

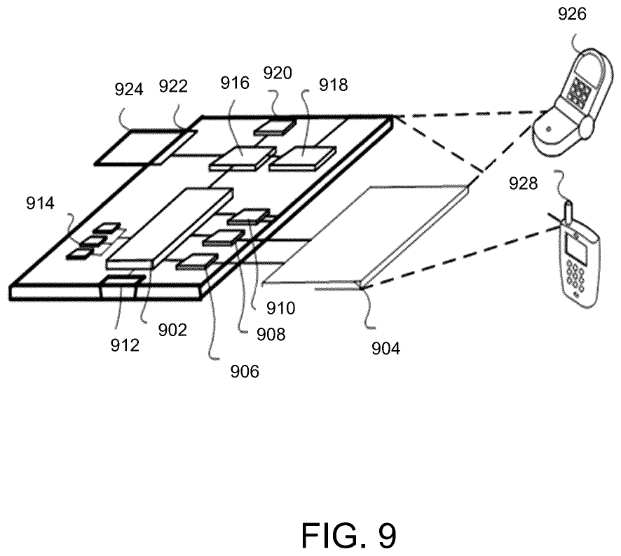

FIG. 9 shows an example of a computing device 900, which may be used with the techniques described in this disclosure. For example, the computing device 900 may be a part of an ultrasound diagnostic system. Computing device 900 includes a processor 902, memory 914, an input/output device such as a display 904, a communication interface 916, and a transceiver 918, among other components. The device 900 may also be provided with a storage device, such as a micro drive or other device, to provide additional storage. Each of the components 900, 902, 914, 904, 916, and 918 are interconnected using various buses, and several of the components may be mounted on a common motherboard or in other manners as appropriate.

The processor 902 may execute instructions within the computing device 900, including instructions stored in the memory 914. The processor may be implemented as a chipset of chips that include separate and multiple analog and digital processors. The processor may provide, for example, coordination of the other components of the device 900, such as control of user interfaces, applications run by device 900, and wireless communication by device 900.

The processor 902 may communicate with a user through the control interface 908 and the display interface 906 coupled to a display 904. The display 904 may be, for example, a thin-film-transistor liquid crystal display (TFT LCD) or an organic light emitting diode (OLED) display, or other appropriate display technology. The display interface 906 may comprise appropriate circuitry for driving the display 904 to present graphical and other information to a user. The control interface 908 may receive commands from a user and convert them for submission to the processor 902. In addition, an external interface 912 may be provided in communication with processor 902, so as to enable near-area communication of device 900 with other devices. External interface 912 may provide, for example, for wired communication in some implementations, or for wireless communication in other implementations, and multiple interfaces may also be used.

The memory 914 stores information within the computing device 900. The memory 914 may be implemented as one or more of a computer-readable medium or media, a volatile memory unit or units, or a non-volatile memory unit or units. Expansion memory 924 may also be provided and connected to device 900 through expansion interface 922, which may include, for example, a single in-line memory module (SIMM) card interface. Such expansion memory 924 may provide extra storage space for device 900, or may also store applications or other information for device 900. Specifically, expansion memory 924 may include instructions to carry out or supplement the processes described above, and may include secure information also. Thus, for example, expansion memory 924 may be provided as a security module for device 900, and may be programmed with instructions that permit secure use of device 900. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 914, expansion memory 924, memory on processor 902, or a propagated signal that may be received, for example, over transceiver 918 or external interface 912.

Device 900 may communicate wirelessly through communication interface 916, which may include digital signal processing circuitry where necessary. Communication interface 916 may, in some cases, be a cellular modem. Communication interface 916 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 918. In addition, short-range communication may occur, such as using a Bluetooth, WiFi, or other such transceiver (not shown). In addition, global positioning system (GPS) receiver module 920 may provide additional navigation-and location-related wireless data to device 900, which may be used as appropriate by applications running on device 900.

Device 900 may also communicate audibly using audio codec 910, which may receive spoken information from a user and convert it to usable digital information. Audio codec 910 may likewise generate audible sound for a user, such as through a speaker, e.g., in a handset of device 900. Such sound may include sound from voice telephone calls, may include recorded sound (e.g., voice messages, music files, etc.) and may also include sound generated by applications operating on device 900.

The computing device 900 may be implemented in a number of different forms, as shown in FIG. 4. For example, it may be implemented as a cellular telephone 926. It may also be implemented as part of a smartphone 928, smart watch, personal digital assistant, or other similar mobile device.

### Operating Environment

Implementations of the subject matter and the operations described in this specification may be implemented in digital electronic circuitry, or in computer software, firmware, or hardware, including the structures disclosed in this specification and their structural equivalents, or in combinations of one or more of them. Implementations of the subject matter described in this specification may be implemented as one or more computer programs, i.e., one or more modules of computer program instructions, encoded on the computer storage medium for execution by, or to control the operation of, data processing apparatus. Alternatively or in addition, the program instructions may be encoded on an artificially generated propagated signal, e.g., a machine-generated electrical, optical, or electromagnetic signal, that is generated to encode information for transmission to a suitable receiver apparatus for execution by a data processing apparatus. A computer storage medium may be, or be included in, a computer-readable storage device, a computer-readable storage substrate, a random or serial access memory array or device, or a combination of one or more of them. Moreover, while a computer storage medium is not a propagated signal, a computer storage medium may be a source or destination of computer program instructions encoded in an artificially generated propagated signal. The computer storage medium may also be, or be included in, one or more separate physical components or media (e.g., multiple CDs, disks, or other storage devices).

The operations described in this specification may be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus may include special purpose logic circuitry, e.g., a field programmable gate array (FPGA) or an application specific integrated circuit (ASIC). The apparatus may also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment may realize various different computing model infrastructures, such as web services, distributed computing and grid computing infrastructures.

A computer program (also known as a program, software, software application, script, or code) may be written in any form of programming language, including compiled or interpreted languages, declarative or procedural languages, and it may be deployed in any form, including as a standalone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language resource), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA or an ASIC.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio or video player, a game console, GPS receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, implementations of the subject matter described in this specification may be implemented on a computer having a display device, e.g., a cathode ray tube (CRT) or liquid crystal display (LCD) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input. In addition, a computer may interact with a user by sending resources to and receiving resources from a device that is used by the user; for example, by sending web pages to a web browser on a user's client device in response to requests received from the web browser.

Implementations of the subject matter described in this specification may be implemented in a computing system that includes a back end component, e.g., as a data server, or that includes a middleware component, e.g., an application server, or that includes a front end component, e.g., a client computer having a graphical user interface or a Web browser through which a user may interact with an implementation of the subject matter described in this specification, or any combination of one or more such back end, middleware, or front end components. The components of the system may be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system may include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) may be received from the client device at the server.

A system of one or more computers may be configured to perform particular operations or actions by virtue of having software, firmware, hardware, or a combination of them installed on the system that in operation causes or cause the system to perform the actions. One or more computer programs may be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the implementations described above should not be understood as requiring such separation in all implementations, and it should be understood that the described program components and systems may generally be integrated together in a single software product or packaged into multiple software products.

Thus, particular implementations of the subject matter have been described. Other implementations are within the scope of the following claims. In some cases, the actions recited in the claims may be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Each numerical value presented herein, for example, in a table, a chart, or a graph, is contemplated to represent a minimum value or a maximum value in a range for a corresponding parameter. Accordingly, when added to the claims, the numerical value provides express support for claiming the range, which may lie above or below the numerical value, in accordance with the teachings herein. Absent inclusion in the claims, each numerical value presented herein is not to be considered limiting in any regard.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention.

Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

What is claimed is:

1. A method for locating an unknown arrangement of ultrasound transducer elements, the method comprising:
acquiring a set of raw ultrasound data using a flexible or rigid array of transducer elements;
extracting a set of features from the ultrasound data, the features being derived from fundamental physics of ultrasound propagation and structural characteristics of a target object being imaged;
organizing and structuring the extracted features as input for a machine learning model; and
determining spatial coordinates of each ultrasound transducer element based on an output of the machine learning model.

2. The method of claim 1, further comprising:
translating the spatial coordinates into a structured grid or matrix that represents a spatial layout of the array of transducer elements.

3. The method of claim 2, further comprising:
performing image reconstruction of the target object based on the raw ultrasound data and the structured grid or matrix that represents the spatial layout of the array of transducer elements.

4. The method of claim 3, further comprising:
performing one or more of log compression, envelope detection, noise reduction and smoothing, dynamic range adjustment for a reconstructed image.

5. The method of claim 1, wherein the machine learning model is trained using a dataset including one or more of synthetic or real ultrasound data.

6. The method of claim 5, wherein the synthetic data is generated using a physics-based simulation of ultrasound propagation under varying spatial configurations and acoustic properties of imaging media.

7. The method of claim 5, wherein the real ultrasound data includes labeled datasets comprising ultrasound data acquired from predefined transducer configurations on different anatomical surfaces, including curved, irregular, and flat surfaces.

8. The method of claim 1, further comprising:
applying time gain compensation (TGC) to the raw ultrasound data prior to feature extraction, wherein TGC compensates for signal attenuation based on travel distance.

9. The method of claim 1, further comprising:
applying envelope extraction to isolate an amplitude envelope of the raw ultrasound data.

10. The method of claim 1, further comprising:
applying bandpass filtering to suppress unwanted frequency components while retaining frequencies that contain pertinent spatial information.

11. The method of claim 1, further comprising:
generating time-of-flight (ToF) data by capturing a time delay between transmitted and received signals for each transducer element pair; and
utilizing the ToF data as an additional input feature for the machine learning model to refine the spatial coordinates.

12. The method of claim 11, wherein the extracted features comprise one or more of:
a difference in first arrival times between adjacent transducer elements derived from the ToF data;
amplitudes of first arriving signals derived from the ToF data;
cross-correlation coefficient derived from the ToF data;
and
cross-correlation lag and corresponding coefficient values between received signals across a plurality of transducer pairs.

13. The method of claim 1, further comprising:
integrating data from previously estimated transducer positions as additional input features for the machine learning model, wherein temporal consistency between frames is utilized to refine the spatial coordinates.

14. The method of claim 1, further comprising:
incorporating image quality metrics derived from previous ultrasound frames as additional input features for the machine learning model, wherein the image quality metrics include one or more of entropy, sharpness, and coherence utilized to refine the spatial coordinates.

15. The method of claim 1, wherein organizing and structuring the extracted features as the input for the machine learning model comprises:
organizing the extracted features into a consistent format to ensure compatibility with the machine learning model.

16. The method of claim 15, wherein organizing and structuring the extracted features as the input for the machine learning model comprises:
embedding spatial relationships as relative distance matrices or adjacency matrices for the machine learning model.

17. The method of claim 1, further comprising:
segmenting the raw ultrasound data to isolate specific regions of interest within the ultrasound data.

18. The method of claim 1, wherein the machine learning model is one of a deep neural network, a convolutional neural network, a recurrent neural network, or a long short-term memory network.

19. The method of claim 1, further comprising:
dynamically adjusts data acquisition parameters based on the spatial coordinates.

20. A system for locating an unknown arrangement of ultrasound transducer elements, comprising:
a processor; and
a memory, coupled to the processor, configured to store executable instructions that, when executed by the processor, cause the processor to perform operations comprising:
acquiring a set of raw ultrasound data using a flexible or rigid array of transducer elements;
extracting a set of features from the ultrasound data, the features being derived from fundamental physics of ultrasound propagation and structural characteristics of a target object being imaged;
organizing and structuring the extracted features as input for a machine learning model; and
determining spatial coordinates of each ultrasound transducer element based on an output of the machine learning model.
